(19) United States
(12) Patent Application Publication
(10) Pub. No.: US 2008/0144851 A1
(43) Pub. Date: Jun. 19, 2008

(54) Method and Device for Controlling Active Noises Using Film Speakers

(76) Inventor: Hoon Kim, Seoul (KR)

Correspondence Address: WELLS ST. JOHN P.S., 601 W. FIRST AVENUE, SUITE 1300, SPOKANE, WA, 99201, US

(21) Appl. No.: 11/885,912

(22) PCT Filed: Mar. 8, 2006
(86) PCT No.: PCT/KR2006/000812

§ 371 Date: Jan. 16, 2008

(30) Foreign Application Priority Data
Mar. 9, 2005 (KR) 10-20050019691

(51) Int. Cl. A61F 11/06

(52) U.S. Cl. 381/71.5

(57) ABSTRACT

Disclosed therein is a method and device for active noise control using film speakers, in which a noise source is approached at the maximum using a film speaker to surround the noise source, thereby most effectively shielding a noise generated from the noise source. This invention implements a device for active noise control using a film speaker, the device comprising: a film speaker adapted to output an cancellation sound for canceling a noise emitted from a noise source, the cancellation sound being a sound having an opposite phase to that of the noise emitted from the noise source; an enclosure for film speaker adapted to shield the external emission of the cancellation sound emitted from the rear side of the film speaker; an input microphone installed at the proximity of the noise source surrounded by the film speaker and adapted to be input with the noise emitted from the noise source; and an active noise control means adapted to generate the opposite phase sound or the cancellation sound corresponding to the noise applied thereto from the input microphone, and control the driving of the film speaker for emission of the opposite phase sound, and revise the residual error from the error microphone.

### BRIEF DESCRIPTION OF DRAWINGS

FIG. 1 is a block diagram illustrating the inner construction of a conventional device for active noise control according to prior art;

FIG. 2 is a schematic diagrammatic view illustrating the construction of a device for active noise control using a film speaker according to the present invention;

FIG. 3 is a block diagram illustrating the construction of an embodiment of an active noise control means shown in FIG. 2; and

FIG. 4 is a flowchart illustrating the process of active noise control using a film speaker according to the present invention.

### TECHNICAL FIELD

The present invention relates to a method and device for active noise control using film speakers, and more particularly, to such a method and device for active noise control using film speakers, in which a noise source is approached at the maximum using a film speaker to surround the noise source, thereby most effectively shielding a noise generated from the noise source.

Among public nuisances, noise problems are very prevalent and difficult to overcome. Noises from household electric appliances, for example refrigerators, washing machines and other noise sources, within buildings, from ventilation duct work, within vehicles, and other sources have been increasingly recognized as problematic areas in recent years. Although various means and materials for controlling noises have been proposed, there have been no satisfactory, versatile materials for attenuating sound.

The choice of a particular sound insulating material for a given application is determined not only by its ability to attenuate sound but by other considerations as well. These considerations include cost, weight, thickness, fire resistance, etc. For example, the technically well known sound attenuating materials include felts, foams, compressed fibers, glass powder or “rock wool,” and recycled fabrics (shoddy materials) which have been hammer milled, resinated, and thermosetting. Unfortunately, there are disadvantages associated with each of these materials. Further, there has been a continuing need for acoustical insulation materials which exhibit superior sound attenuating properties.

Recently, there has been made a research on an active noise canceling method in which the sound same as a noise is formed to have an opposite phase to that of the noise so as to be output to the outside via a speaker so that the noise can be cancelled. In fact, a continuous effort has been made to put a product employing such an active noise canceling method on the market, but such an effort had embraced the difficulty of having to well design an installation space based on the acoustics in order to maximize an active noise cancellation effect.

Conventionally, an active noise cancellation system is generally disposed in a duct-shaped space formed artificially so as to increase a noise cancellation effect.

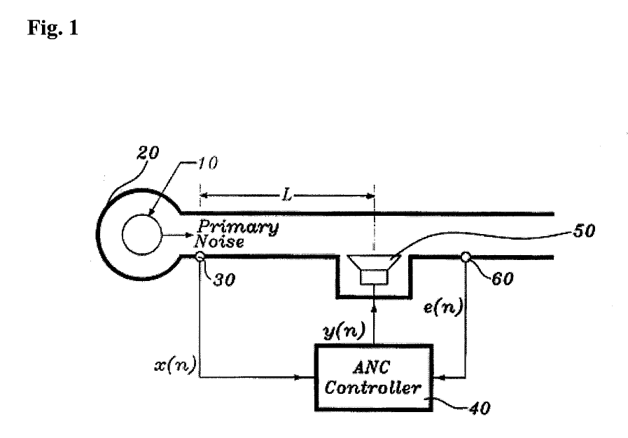

Now, the operation of the conventional active noise canceling device will be described hereinafter with reference to FIG. 1.

FIG. 1 is a block diagram illustrating the inner construction of a conventional device for active noise control according to prior art.

Referring to FIG. 1, a duct 20 is formed around a noise source 10 to surround the noise source 10. A noise data audibly input via an input microphone 30 provided at the proximity of the noise source 10 is converted into a digital data (x(n)) to be input to an ANC (Active Noise Control) controller 40 as a DSP (digital signal processor) chip.

The ANC controller 40 is adapted to process the digital data x(n) applied thereto from the input microphone 30 using FXLMS (Filtered-X LMS) algorithm as an optimal filter algorithm for active noise cancellation so as to generate a noise data having a phase opposite to that of the noise data audibly input via the input microphone 30 to cancel the noise data. Then, the ANC controller 40 converts the generated noise data having the opposite phase into an analog signal y(n) and outputs an cancellation sound through a speaker 50 disposed at an optimal position to cancel the noise.

The filter algorithm for active noise control includes a function for compensating for an algorithm after the remaining noise which is not cancelled completely is audibly input through an error microphone 60.

As well known in the art, it is preferable to approach a noise source to output a cancellation sound so that an effective noise cancellation can be performed. But, in case of using a general speaker, it is not easy to install it after approaching the noise source at the maximum due to a fixed form or enclosure problem, and hence a conventional active noise canceling device has employed a method as the next-best thing in which a space where to cancel a noise is deformed to a duct shape and a speaker is mounted inwardly of the duct from the outside to shield the sound having an opposite phase coming from the rear side of the speaker.

However, such a conventional active noise canceling device has a demerit in that it may be often impossible to modify the noise canceling space to a duct shape. Although it is possible to modify the noise canceling space to the duct shape, much cost is spent and it is difficult to put the active noise canceling device on the market.

### DISCLOSURE OF INVENTION

Accordingly, the present invention has been made to solve the above-mentioned problems occurring in such a conventional active noise canceling device, and it is an object of the present invention to provide a method and device for active noise control using film speakers, in which a noise source is approached at the maximum using a film speaker to surround the noise source, thereby most effectively shielding a noise generated from the noise source.

To accomplish the above object, according to one aspect of the present invention, there is provided a device for active noise control using a film speaker, the device including:

a film speaker adapted to output a cancellation sound for canceling a noise emitted from a noise source, the cancellation sound being a sound having an opposite phase to that of the noise emitted from the noise source;

an enclosure for film speaker adapted to shield the external emission of the cancellation sound emitted from the rear side of the film speaker;

an input microphone adapted to be input with the noise emitted from the noise source; and

an active noise control means adapted to generate the opposite phase sound or the cancellation sound corresponding to the noise applied thereto from the input microphone, and control the driving of the film speaker for emission of the opposite phase sound.

### BEST MODE FOR CARRYING OUT THE INVENTION

Now, an explanation on the preferred embodiments of the present invention will be in detail given hereinafter with reference to the accompanying drawings. It will however be obvious to a person skilled in the art that the present invention is not limited to or by the embodiments.

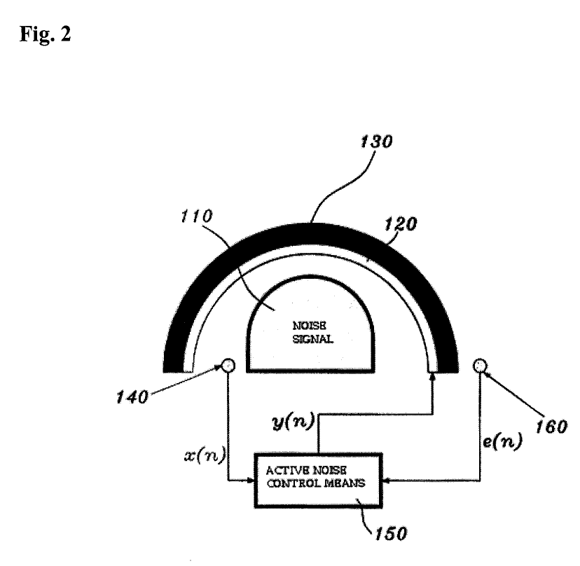

FIG. 2 is a schematic diagrammatic view illustrating the construction of a device for active noise control using a film speaker according to the present invention.

Referring to FIG. 2, a reference numeral 110 denotes a noise source, a reference numeral 120 denotes a film speaker installed at the proximity of the noise source 110 for surrounding the noise source 110, a reference numeral 130 denotes an enclosure for film speaker installed at a rear surface of the film speaker 120 for shielding a sound having an opposite phase to that of a noise emitted from the noise source, the sound being emitted from the rear side of the film speaker 120, and a reference numeral 140 denotes an input microphone installed at the proximity of the noise source 110 surrounded by the film speaker 120 for being input with the noise emitted from the noise source 110.

Also, a reference numeral 150 denotes an active noise control means for generating a sound (cancellation sound) having an opposite phase to that of the noise applied thereto from the input microphone 140, converting the generated opposite phase sound into an analog signal, driving the film speaker 120 to cancel the noise, and being input with a remaining noise existing outside the enclosure 130 for film speaker through an error microphone 160 to correct the generated opposite phase sound.

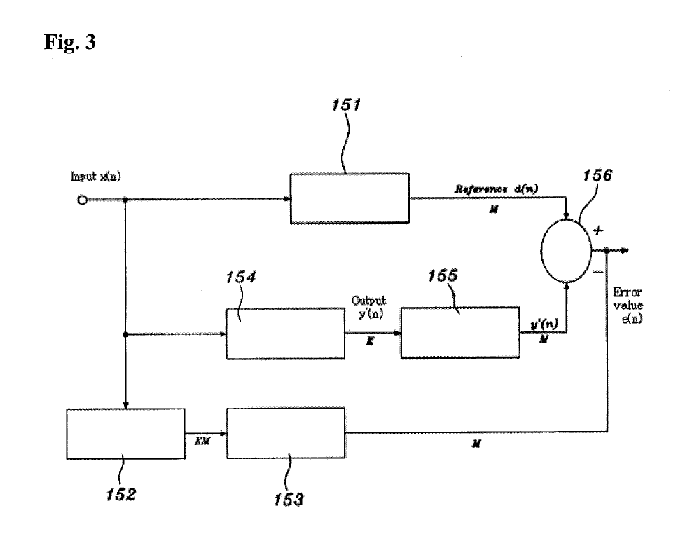

As shown in FIG. 3, the active noise control means 150 includes a first path value transferring unit 151 for transferring a noise value (reference d(n)) on a first path, which is generated when a noise x(n) reaches an error microphone 160, a second path value transferring unit 155 for transferring a signal value on a second path, which is generated when an output signal from the film speaker 120 reaches the error microphone 160, an error value generator 156 for outputting an error value e(n) of the error microphone 160 which is re-input with a remaining noise which is substantially not cancelled completely, a second path value approximator (Ŝ) 152 for internally pre-calculating the noise value on the second path so as to update a coefficient value of an adaptive filter based on the effect of the second path, a filter coefficient value updator 153 for updating a filter coefficient value using an LMS algorithm for minimizing an error between an output signal of a linear filter and a desired signal, and an adaptive filter W(n) 154 for generating a signal having an opposite phase to that of the noise x(n) generated from the noise source depending on the filter coefficient value generated from the filter coefficient value updator 153.

The operation of the active noise controlling device using a film speaker as constructed above will be described hereinafter in detail.

First, when a noise is generated from the noise source 110, the input microphone 140 is input with a noise value corresponding to the noise for application to the active noise control means 150. Then, the active noise control means 150 converts the input signal indicative of the noise value into a signal having an opposite phase to that of the input signal to output the converted signal via the film speaker 120 so that the noise generated from the noise source 110 can be canceled out.

That is, the active noise control means 150 generates a signal y′(n) having an opposite phase to that of the input signal through the adaptive filter W(n) 154 for converting an actual input signal x(n) into the signal having an opposite phase to that of the input signal by applying an LMS algorithm for minimizing an error between an output signal of the adaptive filter 154 and a desired signal, and outputs the signal y′(n) through the film speaker 120 via the second path value transferring unit (S) 155 on a second path, which is generated when reaching the error microphone 160 so that the noise generated from the noise source can be canceled out.

At this time, since the enclosure 130 for film speaker installed at a rear surface of the film speaker 120 should output only a noise having an opposite phase to that of an existing noise to cancel the existing noise, it is possible to effectively control an opposite phase sound emitted from the rear surface of the film speaker 120.

In the meanwhile, a remaining noise e(n) which is not canceled completely through the film speaker 120 is re-input to the error microphone 160 and then the active noise control means 150. The remaining noise e(n) is applied to the LMS algorithm along with an approximated second path (Ŝ) value obtained by internally pre-calculating the noise value so as to reduce the affect of the second path (S), so that respective parameters is fine-adjusted to thereby apply a next noise more accurately to the adaptive filter W(n) 154.

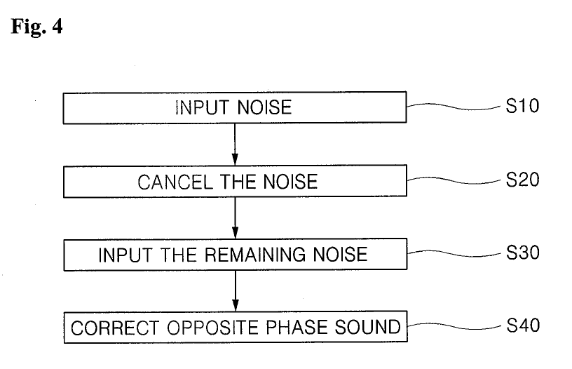

FIG. 4 is a flowchart illustrating the process of canceling an active noise using a film speaker according to the present invention.

Referring to FIG. 4, the active noise controlling method includes the following steps of: a step (S10) of allowing an input microphone installed at the proximity of the noise source surrounded by the film speaker to be input with the noise emitted from the noise source; a step (S20) of generating a sound (cancellation sound) having an opposite phase to that of the input noise, converting the generated opposite phase sound into an analog signal, and canceling the noise via the film speaker; a step (S30) of allowing an error microphone to be input with a remaining noise existing outside the enclosure for film speaker installed at a rear surface of the film speaker; and a step (S40) of correcting the generated opposite phase sound depending on the remaining noise input to the error microphone.

Typically, in case of a conventional existing active noise controlling device, it is difficult for an existing speaker to shield a noise source acoustically diffused in all directions. Thus, as shown in FIG. 1, a surrounding space where to cancel a noise is formed in a duct shape to allow a noise to be transferred via one place only, and a speaker is mounted inside the duct to thereby reduce the noise. In this case, it is impossible to form all the noise source spaces in a duct shape as well as much cost is spent.

In the meantime, a system applied to the method and device for active noise control using a film speaker is implemented in such a fashion as to encircle the surrounding space of a noise source using a special-shaped speaker which is called a film speaker, thereby effectively reducing a noise without deforming the noise source space and greatly decreasing the cost accordingly.

### INDUSTRIAL APPLICABILITY

As described above, according to the present invention, a noise source is encircled using a special speaker called a film speaker so as to most effectively shield a noise of the noise source, thereby effectively interrupting the noise acoustically diffused in all directions.

In addition, since an existing surrounding space of the noise source does not need to be deformed to a duct shape, the cost can be saved significantly.

While the present invention has been described with reference to the particular illustrative embodiments, it is not to be restricted by the embodiments but only by the appended claims. It is to be appreciated that those skilled in the art can change or modify the embodiments without departing from the scope and spirit of the present invention.

What is claimed is:

1. A device for active noise control using a film speaker, the device comprising:
a film speaker adapted to output an cancellation sound for canceling a noise emitted from a noise source, the cancellation sound being a sound having an opposite phase to that of the noise emitted from the noise source;
an enclosure for film speaker adapted to shield the external emission of the cancellation sound emitted from the rear side of the film speaker;
an input microphone adapted to be input with the noise emitted from the noise source; and
an active noise control means based on a DSP (Digital Signal Processor) adapted to generate the opposite phase sound or the cancellation sound corresponding to the noise applied thereto from the input microphone, and control the driving of the film speaker for emission of the opposite phase sound.

2. The device as claimed in claim 1, wherein the film speaker is installed at the proximity of the noise source and adapted to surround the noise source.

3. The device as claimed in claim 1, wherein the enclosure for film speaker is installed at a rear surface of the film speaker.

4. The device as claimed in claim 1 further comprising an error microphone adapted to be input with a remaining noise existing outside the enclosure for film speaker to transfer the input remaining noise to the active noise control means.

5. The device as claimed in claim 1 or 4, wherein the active noise control means comprises:
a first path value transferring unit adapted to transfer a noise value on a first path, which is generated when the noise emitted from the noise source reaches an error microphone;
a second path value transferring unit adapted to transfer a signal value on a second path, which is generated when an output signal from the film speaker reaches the error microphone;
an error value generator adapted to perform subtraction between the first path value and the second path value, and output a result value of the subtraction as an error value of the error microphone which is re-input with a remaining noise which is substantially not cancelled completely;
a second path value approximator adapted to proximate the noise value on the second path so as to update a coefficient value of an adaptive filter based on the effect of the second path;
a filter coefficient value updator adapted to update a filter coefficient value using an LMS algorithm for minimizing an error between an output signal of a linear filter and a desired signal; and
an adaptive filter adapted to update the filter coefficient value under the control of the filter coefficient value updator, and generate a signal having an opposite phase to that of the noise generated from the noise source to cancel the noise.

6. The device as claimed in claim 1 or 4, wherein the active noise control means is further adapted to be input with a remaining noise existing outside the enclosure for film speaker through an error microphone to correct the generated opposite phase sound.

7. A method of active noise control using a film speaker, the method comprising the steps of:
a step (S10) of allowing an input microphone installed at the proximity of the noise source surrounded by the film speaker to be input with the noise emitted from the noise source;
a step (S20) of generating a sound (cancellation sound) having an opposite phase to that of the input noise, converting the generated opposite phase sound into an analog signal, and canceling the noise via the film speaker;
a step (S30) of allowing an error microphone to be input with a remaining noise existing outside the enclosure for film speaker installed at a rear surface of the film speaker; and
a step (S40) of correcting the generated opposite phase sound depending on the remaining noise input to the error microphone.

8. The method as claimed in claim 7, wherein the step (S40) comprises correcting the generated opposite phase sound in such a fashion as to correct a filter value of LMS algorithm for generating the opposite phase sound.
